(19) United States
(12) Patent Application Publication
(10) Pub. No.: US 2023/0255567 A1
(43) Pub. Date: Aug. 17, 2023

(54) DEVICES AND METHODS FOR COLLECTING INTEROCEPTION DATA

(71) Applicant: HUMA THERAPEUTICS LIMITED, London, Greater London (GB)

(72) Inventors: Davide Morelli, London (GB); David Plans, London (GB); Sonia Ponzo, London (GB); Geoff Bird, London (GB)

(21) Appl. No.: 18/002,997

(22) PCT Filed: Jul. 13, 2021
(86) PCT No.: PCT/EP2021/069541

§ 371 Date: Dec. 22, 2022

(30) Foreign Application Priority Data
Jul. 14, 2020 (GB) 2010855.1

(51) Int. Cl. A61B 5/00

(52) U.S. Cl. CPC A61B 5/7415; A61B 5/486; A61B 5/7435; A61B 5/7475

(57) ABSTRACT

A system, and related methods, for estimating a level of interoception in a subject, the system including a heartbeat sensor for sensing the subject's heartbeat as manifested locally to the sensor, a user interface for receiving input from the subject and a processor configured to: receive information representing the timing of sensed heartbeats; form, in dependence on the received information, a periodic output having the same frequency as the sensed heartbeats; present the periodic output to the subject; receive input of a first type from the user interface; and in dependence on the input of a first type, adjust the phase with which the periodic output is to presented to the subject.

### RELATED APPLICATION

This application claims priority to and the benefit of GB application number GB 2010855.1 filed Jul. 14, 2020, the disclosure of which is incorporated herein in its entirety.

### FIELD OF THE INVENTION

Embodiments of the disclosed invention relate to collecting interoception data from a subject. Interoception is the sensing by a subject of their own physiological condition.

### BACKGROUND

During the past century, studies have been made of subjects' levels of interoception. These studies have led to an understanding that the acuity of a subject's interoception can be helpful in assessing the subject's wellbeing. For example, disrupted interoceptive processing has been found to be a feature of many psychiatric disorders including anxiety, panic disorder, depression and eating disorders. See, e.g., Khalsa, Sahib S., Ralph Adolphs, Oliver G. Cameron, Hugo D. Critchley, Paul W. Davenport, Justin S. Feinstein, Jamie D. Feusner, et al. 2018. “Interoception and Mental Health: A Roadmap.” Biological Psychiatry. Cognitive Neuroscience and Neuroimaging 3 (6): 501-13; Barrett, Lisa Feldman, Karen S. Quigley, and Paul Hamilton. 2016. “An Active Inference Theory of Allostasis and Interoception in Depression.” Philosophical Transactions of the Royal Society of London. Series B, Biological Sciences 371 (1708): 20160011; and

Paulus, Martin P., and Murray B. Stein. 2010. “Interoception in Anxiety and Depression.” Brain Structure & Function 214 (5-6): 451-63.

Some examples of interoceptive pathways that have been studied are cardiovascular (see, e.g., Critchley, Hugo D., Stefan Wiens, Pia Rotshtein, Arne Öhman, and Raymond J. Dolan. 2004. “Neural Systems Supporting Interoceptive Awareness.” Nature Neuroscience.; and Garfinkel, Sarah N., Anil K. Seth, Adam B. Barrett, Keisuke Suzuki, and Hugo D. Critchley. 2015. “Knowing Your Own Heart: Distinguishing Interoceptive Accuracy from Interoceptive Awareness.” Biological Psychology 104 (January): 65-74), nociceptive (see, e.g., Mohr, Mariana von, Mariana von Mohr, Charlotte Krahé, Brianna Beck, and Aikaterini Fotopoulou. 2018. “The Social Buffering of Pain by Affective Touch: A Laser-Evoked Potential Study in Romantic Couples.” Social Cognitive and Affective Neuroscience. https://doi.org/10.1093/scan/nsy085.) and autonomic responses such as temperature sensation, and feelings of thirst and hunger. Each of the references noted above is incorporated by reference in its entirety.

Cardiovascular interoception relates to a subject's ability to sense their own cardiac rhythm. In the past this has been evaluated by techniques such as forming a synthesised sound having the same frequency as a subject's heart rate, playing that sound back to the subject at a range of phase offsets from the subject's heartbeats, and asking the subject to identify the play-back that is synchronised with their heartbeats. This technique has several disadvantages. First, in order to know the timing of the subject's heartbeats it is normally necessary to use equipment such as an electrocardiograph machine, which is typically bulky, relatively non-portable and generally unsuitable for use outside a clinical setting. Second, the phase of the cardiac rhythm varies around the body due to the time taken for cardiac pressure waves to propagate through the body. It has been found that some people do not sense their cardiac rhythm at their heart, but at some other location in the body. From the test described above, those people might be considered to have poor cardiovascular interoception because they perceive synchronisation to occur at a significant offset from their heartbeats.

There is a need for a better way of estimating cardiac interoception.

### SUMMARY

In an aspect, embodiments of the invention relate to a system for estimating a level of interoception in a subject, the system including a heartbeat sensor for sensing the subject's heartbeat as manifested locally to the sensor, a user interface for receiving input from the subject and a processor configured to: receive information representing the timing of sensed heartbeats; form, in dependence on the received information, a periodic output having the same frequency as the sensed heartbeats; present the periodic output to the subject; receive input of a first type from the user interface; and in dependence on the input of a first type, adjust a phase with which the periodic output is to presented to the subject.

One or more of the following features may be included. The processor may be further configured to: receive input of a second type from the user interface, and in response to the input of the second type to store a current timing offset between the sensed heartbeats and periodic elements of the periodic output.

The processor may be further configured to, prior to presenting the periodic output to the subject, determine a random offset from the sensed heartbeats and cause the periodic output to be initially presented to the subject at the phase corresponding to an offset between the sensed heartbeats and periodic elements of the periodic output that is equal to the random offset.

The periodic output may be a sound including periodic elements, such as tones.

The user interface may provide a first user interface element that can be adjusted continuously by a user. The first user interface element may form the input of the first type.

The first user interface element may be a wheel. It may be a physical wheel or a rotatable element displayed on a touch screen, the representation of which is movable in response to touch interaction therewith by a user.

The second user interface element may be a button. It may be a physical button or a button displayed on a touch screen.

The system may be configured to repeatedly perform the steps set out above to form an estimate of the consistency of the stored timing offsets.

The system may be configured to form an estimate of the subject's level of interoception in dependence on one or both of (i) the absolute values of one or more of the stored timing offsets and (ii) the estimated consistence of the stored timing offsets.

The system may be configured to assess the identity of the subject in dependence on one or both of (i) the absolute values of one or more of the stored timing offsets and (ii) the estimated consistence of the stored timing offsets.

In another aspect, embodiments off the invention relate to a method for estimating a level of interoception in a subject, the method including: sensing the timing of the subject's heartbeats as manifested locally to a sensor; forming, in dependence on the received information, a periodic output having the same frequency as the sensed heartbeats; presenting the periodic output to the subject; receiving input of a first type from a user interface; and in dependence on the input of a first type, adjusting the phase with which the periodic output is to presented to the subject.

### BRIEF DESCRIPTION OF DRAWINGS

Embodiments of the present invention will now be described by way of example with reference to the accompanying drawings. In the drawings:

FIG. 1 is a schematic diagram of a system for capturing interoception data, in accordance with an embodiment of the invention.

FIG. 2 is a schematic diagram of a user interface for input of interoception data, in accordance with an embodiment of the invention.

FIG. 3 is a graph illustrating probability density functions of simulated participants' behaviour and real participants' answers, in accordance with an embodiment of the invention.

### DETAILED DESCRIPTION

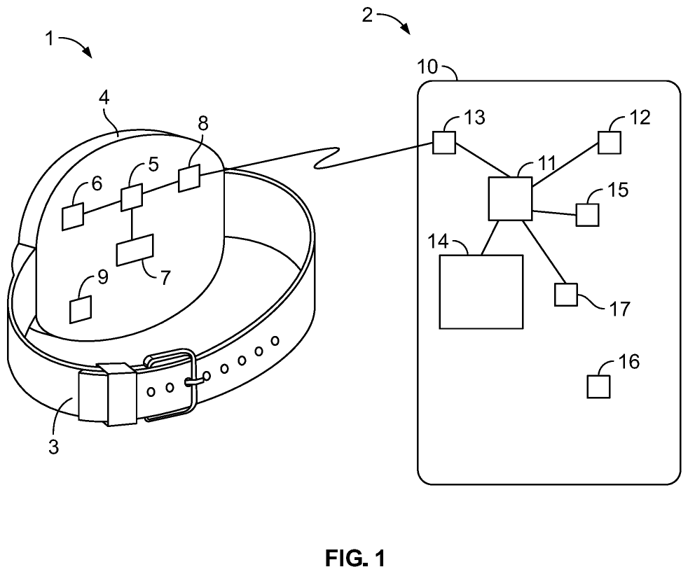

Referring to FIG. 1, a wearable heart monitor device 1 may be configured to communicate with a smartphone 2.

The wearable device includes a strap 3 and a housing 4. The housing includes a processor 5, a memory 6, a pulse sensor 7, and a wireless transceiver 8. The wearable device is powered by a battery 9. The memory 6 stores in non-transient form instruction code executable by the processor 5 so that the wearable device can perform the functions described herein. The pulse sensor 7 is exposed at the interior-facing surface of the device 1. The pulse sensor, also referred to herein as a heartbeat sensor, is capable of sensing the pulse, i.e., the heartbeat, of a subject wearing the device. The pulse sensor may, for example, be an optical pulse sensor. Other pulse-sensing technologies may be used, for instance electrical sensing. The strap 3 is sized for encircling a portion of the subject's body. It may, for example, be a wrist strap. The processor 5 is communicatively coupled to the pulse sensor 7 and the wireless transceiver 8. The wearable device 1 may, for example, be a smart watch, sports tracker, or a dedicated heart rate monitor.

The smartphone 2 includes a housing 10, a processor 11, a memory 12, a first, relatively local transceiver 13, a touch-sensitive display 14, a cellular telephony transceiver 15, a battery 16, and a loudspeaker 17. The smartphone is powered by a battery 16. The memory 12 stores in non-transient form instruction code executable by the processor 11 so that the smartphone can perform the functions described herein. The processor 11 is communicatively coupled to the local transceiver 13, the display 14, and the cellular radio transceiver 15. The local transceiver 13 is a wireless transceiver capable of communicating with wireless transceiver 8 of the wearable device. The touch-sensitive display 14 can display information under the control of the processor 11. The display can also collect touch input from a user making contact with the display and can pass information defining that input to the processor 11. In other embodiments the smartphone may have an input device such as a keyboard or a trackpad that is separate from the display.

The transceivers 8 and 13 operate using a common protocol. That protocol may be a wired protocol such as a universal serial bus (USB), in which case a cable may be used to connect device 1 and smartphone 2. Alternatively, as noted above, the protocol may be a wireless protocol such as Bluetooth or IEEE 802.11. The transceivers can communicate data between each other using that protocol.

In operation, a subject positions the wearable device so that it can sense the subject's pulse. Conveniently the subject may wear the device for this purpose, e.g., around the subject's wrist. The wearable device senses the subject's pulse using pulse sensor 7. The sensor 7 may sense changes in vascular pressure adjacent the sensor, the changes being associated with heartbeats. Accordingly, the sensor can sense the timing of the subject's heartbeats as manifested near the device 1, and hence the intervals between beats. The frequency of the subject's heartbeat can be determined from the interval between beats. The processor 5 of the wearable device 1 receives pulse information from sensor 7 and causes transmitter 8 to transmit an indication of the subject's pulse to the smartphone 2. The processor may transmit the time of each heartbeat (as manifested locally to the device 1). From that the smartphone 2 can determine the timing and frequency of the heartbeats. Alternatively the device 1 may locally determine the timing and frequency of the heartbeats and may transmit that information to the smartphone 2. Other mechanisms are possible. The timing of heartbeats as detected by the device 1 is not necessarily synchronous with the times when the subject's heartbeats because the device 1 may be sensing heartbeats at some distance from the subject's heart. However, the offset between heartbeats as manifested at the device and the times when the subject's heartbeats can be expected to be constant if the device remains at a constant location on the subject's body, and hence the heart rate as sensed by device 1 can be expected to be representative of the subject's heart rate

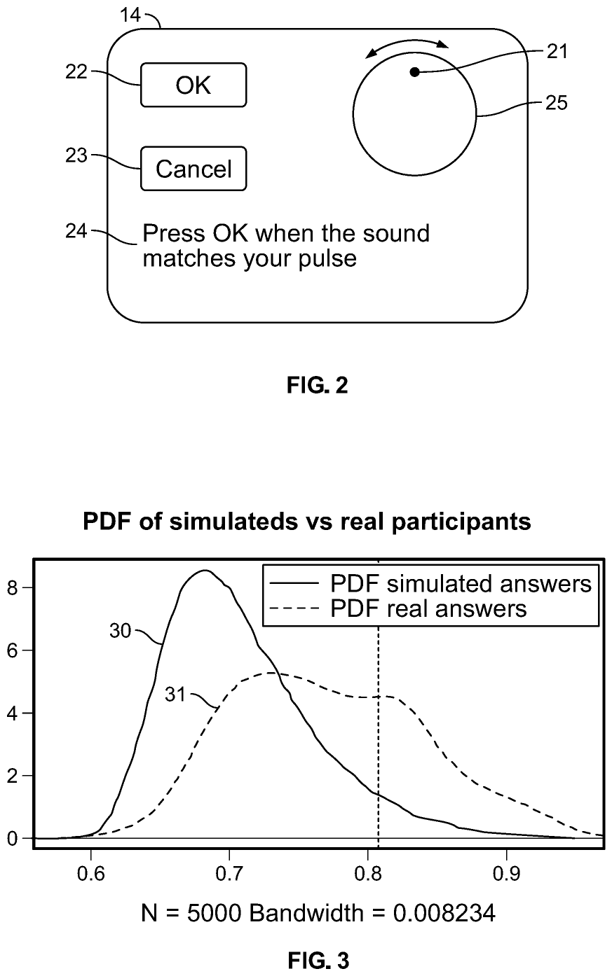

In the manner described above, the processor 11 of the smartphone has knowledge of the subject's heart rate, and the timing of heart pulses at the device 1. The processor 11 forms a sound that contains a rhythm of the same frequency as the indicated pulse rate. The processor 11 then plays the rhythmical sound to the subject by means of loudspeaker 17. At the same time the processor causes the display 14 to present to the subject a user interface. An example of such a user interface is shown in FIG. 2 and will be described below. The display includes one or more user interface elements 25 by which the subject can signal to the processor 11 to change the phase of the sound, by advancing or retarding the sound as played back. The frequency of the rhythm embodied in the sound remains unchanged in response to the subject's input, although it may optionally change as a result of the subject's heart rate changing during the procedure. The subject has been instructed (e.g., by a message 24 on the display 14) to indicate to the smartphone when they sense that the rhythm of the played-back sound is synchronised with their pulse as they interoceptively perceive it. To do this the subject adjusts the rhythm-shift interface element 25 to change the phase of the rhythm embodied in the sound, and at an appropriate time the subject signals to the smartphone that adjustment of the phase is complete. They can do this by interacting with a selection user interface element 22 of the user interface. In response to actuation of the selection element 22, the smartphone gathers information about the subject's adjustment of the rhythm phase. That information may be used as described below.

Processor 11 may form the rhythmical sound in various ways. In one example, data defining a sound suitable for constituting as a single rhythmic feature, for example a beep, may be stored in memory 12. The processor may form the synthesised sound by stringing multiple instances of that rhythmic feature together at a time spacing determined so that the rhythmic features occur at the same periodicity as the sensed cardiac period of the subject. In a second example, memory 12 may store a sound containing multiple successive rhythmic features. The processor may form the sound to be played out by stretching or compressing the stored sound in time so that the rhythmic features occur at the required time spacing. In a further example, the processor 12 may synthesise the rhythmical sound in a purely algorithmic manner. The sound may be formed as it is being played out or in advance of play-out.

When the rhythmical sound is first played out by the processor, it is preferably at a random time offset from the subject's heartbeats as sensed by device 1. To achieve this, the processor may determine a random time offset before the sound is played out, and initially play the sound with the rhythm features of the sound at that determined offset from the sensed heartbeats. The random offset may be determined in the range from 0 seconds to the period of the subject's cardiac rhythm.

Referring to FIG. 2, a user interface suitable for implementing the procedure described above may include a time adjustment element 25 and a selection element 22. The time adjustment element includes a wheel that is rotatable by a user, e.g., by rotating a point 21 on the wheel. Clockwise rotation of the wheel signals the processor to advance the played-out sound. Anti-clockwise rotation of the wheel signals the processor to retard the played-out sound. Other user interface elements may be used for the same purpose, for example a slider or separate advance/retard buttons. Conveniently the time adjustment is continually adjustable. Conveniently, movement of the element (which may be movement of the element as displayed) may cause adjustment of the phase of the played-out sound. Conveniently, movement in one direction may cause the phase to be advanced, and movement in the other direction may cause the phase to be retarded. Selection element 22 allows the subject to indicate when they are happy with the timing of the sound. A cancellation element 23 allows the user to end the procedure. In another arrangement, the phase of the played-out sound may be adjusted automatically, not under control of the subject, and the subject is instructed to activate the selection element when they sense that the phase is synchronised with their heartbeats.

The procedure described above represents a single cardiac interoception test. In practice, multiple tests may be conducted in a single session, and the results aggregated. An overall test process may be as follows:
1 The processor 11 causes the smartphone 2 to begin an interoception assessment for the subject. The interoception assessment includes one or more tests conducted one after the other.
2. Each test proceeds as follows:
   a. The pulse sensing device 1 signals the smartphone 2 with information about the subject's pulse. This signalling may continue during the test. If the subject's pulse rate changes during the test then the smartphone may change the rhythmic frequency of the played-out sound.
   b. The processor forms the sound to be played out in dependence on the detected pulse rate of the subject. The rhythmic features in the sound are at the frequency of the subject's pulse events.
   c. The processor selects a random offset between the timing of pulse events as detected by the sensor 7 and the timing of rhythm features in the sound to be played out. That offset represents the phase of the sound.
   d. The processor 11 causes loudspeaker 17 to play the sound with the selected phase. The subject can provide input to the processor using time adjustment element 25, in response to which the processor alters the phase of the played-out sound.
   e. When the subject is happy that the phase of the played-out sound matches their pulse, they operate selection element 22.
   f. The processor records the timing difference when the selection element 22 is actuated between the timing of pulse events as detected by the sensor 7 and the timing of rhythm events in the played-out sound. Since the pulse events and the rhythm events are cyclical, the processor may record the timing difference in a predetermined direction (e.g., from a pulse event to a rhythm event) or may record the smallest difference when measured in either direction. The recorded difference may be stored in memory 12.
3. Steps a to f are repeated for each test. Then the results are aggregated to estimate the consistency with which the subject estimates offset. The processor may estimate an indication of the consistency between the timing offset recorded from each test. This may, for example, be the timing offset deviation within which a predetermined percentage (for example 80%) of the recorded timing offsets fall. Alternatively it may be the standard deviation of the gathered timings from the tests. The processor may estimate an aggregated value for the timing offsets. This may, for example, be the mean of the recorded timing offsets. If only one test is performed, then the aggregate values may be taken over a single test. Thus, at the end of one or more tests the processor may have (i) an indication of the offset or aggregated offset with which the subject estimates timing offset and/or (ii) an indication of the consistency with which the subject estimates offset.

These indications may, for example, be used as follows. One or both of the indications may be taken as an indication of the subject's wellbeing. For example, an increased level of consistency in estimating the timing offsets may be taken as an indication of the subject's level of interoception. This may present an indication that may assist in assessing the subject's propensity to psychiatric disorders.

In another embodiment, one or both of the indications may be used to identify or authenticate the subject, or to help do so. Because different subjects have different levels of interoception and perceive their pulse at different offsets from their heartbeats, the indications may be characteristic of a specific subject. In one exemplary application, the indications may be used to supplement other identification or authentication data, for example other bio-sensed data about the subject or an entered password.

To allow the indications to be used, for example as described above, the results of the testing may be processed locally to the smartphone, may be displayed to a user of the smartphone, or may be transmitted by the smartphone to another location for analysis, e.g., by a clinician.

Estimating the subject's interoception in this way can provide a number of advantages. First, because the indication of consistency is independent of the offset between the timing of pulse events as detected by the sensor 7 and the timing of the subject's actual heartbeat, the subject's interoception can be estimated without the need for equipment that can sense the timing of the subject's heartbeat. This makes it easier to perform the method described above in nonclinical settings. Second, because the indication of consistency is unrelated to the timing of the subject's actual heartbeat, the indication of consistency can provide valid information for subjects who sends the pulse at a timing offset to their actual heartbeat.

The equipment used to perform the processes described above may be varied. For example, the sensor device 1 and the smartphone 2 may be integrated into a single device. The device referred to above as a smartphone may be embodied as another device, for example a dedicated interoception-sensing device. Instead of playing the sound through a loudspeaker, the sound may be played through headphones. Instead of using a sound to present the rhythmical information, the system may present the rhythmical information in another way, for example visually (e.g., by displaying a pulsating shape or a flashing light or in a mechanical way). Each of the processor mentioned above may be implemented by one or more individual microprocessor devices.

In a test of the system described above, 182 healthy participants (105 females, age range: 18-63, M=26.41, SD=7.58 years), were recruited via an online platform. Participants with psychiatric or neurological history or pregnancy at the time of testing, and current diagnosis of a mental health disorder were excluded.

Instead of focusing on whether participants are able to accurately match a fixed delay between a sound and their own heartbeat, the study participants were allowed to select their preferred delay. Specifically, participants were asked to match a sonified version of their own heart rate, presented at a random (specifically, pseudo-random) delay, by adjusting such delay until they felt it to be in synchrony with their perceived heartbeat. This type of design may provide the advantage of abandoning the concept of accuracy, i.e., whether a participant can correctly identify an exteroceptive stimulus being in synchrony with their own heartbeat, and focusing on consistency instead. Specifically, a participant is considered interoceptive if the consistency between the chosen delays throughout the task is high, i.e., if the delay is similar between trials to a level greater than a predetermined threshold.

The following approach was implemented. The measured heartbeat was sonified. Then the timestamp of this heartbeat was used as the starting point to calculate the average by adding the duration of the current heartbeat to the timestamp which was then used to predict the next one. Specifically, the timestamp of the next heartbeat in seconds was equal to the timestamp of the current heartbeat+60/heart rate in beats per minute (bpm). A pre-stored beep/tone was then played at the timestamp of the next heartbeat with a random delay added. Given that the instantaneous heart rate is close to this calculation, which is performed every 3 seconds, loss in accuracy is considered negligible.

The sonification of the heart rate allows comparison between participant's heart rate and the delayed sonification. Given that the relationship between the phase of the sonified heart rate and the delay chosen by the participant is not linear, consistency is preferably calculated using a periodic function. If, for instance, a participant completes a first trial at 60 bpm and selects a delay of −100 milliseconds (ms), then does another trial at 60 bpm and selects a delay of 900 ms, the dispersion between the two trials would be high and participant's interoceptive consistency would be low. However, −100 ms and 900 ms are the same value if the period is 1000 ms, suggesting the participant is being highly consistent with herself.

Adopting such a circumference approach allows bypassing the issue of similar magnitudes of delay being interpreted as different outcomes (i.e., high vs low interoceptive consistency). If the chosen delay (d) is calculated as a periodic function of the period (p), then a delay of 0 is equal to the period (p). The delay d is an arc that starts from the angle 0 (the point where the circle with centre in the origin and radius r crosses the x axis) when the delay is equal to 0 ms, and does a full circle when d is equal to the period p. The circumference has radius r=p and 2πd and the angle under the arc is equal to α=2πd/p. This formalisation allows one to look at angles to define whether a participant is consistently declaring a similar delay across trials. Consistency is then computed by expressing each angle as a complex number of modulus=1 and argument=α, so that consistency

$$( α ) = \frac{1}{n} mod {\sum }_{j = 1}^{n} {e}_{j}^{i α} .$$

If angles are close to one another, the modulus of their average is close to 1; conversely, if they are randomly positioned on the unitary circle, the modulus is close to 0.

Given that the period changes as a function of changes in heart rate, each delay is be mapped onto a standardised space that can account for such changes. Specifically, in order to obtain one descriptive measure of consistency that is not influenced by changes in periods, each delay's modulus can be mapped onto a circumference of reference, used to map all delays for all participants. To ensure consistency this is calculated using the same radius (r=1) for all participants, the j-th delay αj is mapped onto an angle \hat{α}ⱼ under the same arc on the unitary circle.

On this basis this consistency formula can be considered to be:

$$consistency ( α ) = \frac{1}{n} mod {\sum }_{j = 1}^{n} {e}_{j}^{2 i π nd}$$

$$with {d}_{j} = \frac{{p}_{j} {α}_{j}}{2 π} and {α}_{j} = \frac{2 π {d}_{j}}{{p}_{j}} ,$$

$$thus = 2 π {d}_{j} = \frac{2 π {p}_{j} {α}_{j}}{2 π} = {p}_{j} {α}_{j} .$$

Simulations have been conducted to determine the impact of the number of trials on expected consistency. Results indicated that 20 trials constitute a good compromise between task duration and expected consistency profile, such that the risk of false negatives is minimised. Higher or lower numbers may be used.

A mobile app running on a smartphone was used to implement the process described above. Instead of a separate sensor device 1, the smartphone was configured to detect heart pulse rate using a camera-driven photoplethysmogram sensor to read the heart rate pulse by asking participants to place their finger over the camera. The expected pulse arrival time was around 200 ms at rest.

In addition to gathering data as described above, participants were invited to indicate on a picture of a body where they felt they perceived their heartbeat.

Simulated consistency values from 5000 participants showed a normal distribution with its centre around 0.7 (FIG. 3, line 30). Experimentally, the distribution of consistency values from real participants was binomial (FIG. 3, line 31) and only partially overlapping with the simulated line. To statistically compare whether the two distributions, i.e., the random and real participants' consistency values, were significantly different, a Wilcoxon-signed rank test was performed. Results showed that the participants' answers significantly differed from simulated random answers (Z=10.59, p<0.0001, r=0.15), thus suggesting that real participants' answers were not random.

To test the null hypothesis that the current task does not measure interoception and that participants' answers are random, a threshold value based on the simulated distribution was defined, such that participants with consistency values higher than 95% would be classified as interoceptive with p=0.05. Using this threshold 40 out of 124 participants were labelled as interoceptive, in line with literature findings that approximately ⅓ of healthy participants are accurately able to correctly discriminate and classify interoceptive stimuli. See, e.g., J. Brener and C. Ring, “Towards a psychophysics of interoceptive processes: The measurement of heartbeat detection,” Philosophical Transactions of the Royal Society B Biological Sciences 371 (1708): 20160015 (October 2016), incorporated by reference in its entirety.

Embodiments of the invention may include any individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. Aspects of the present invention may include any such individual feature or combination of features. In view of the foregoing description it will be evident to a person skilled in the art that various modifications may be made within the scope of the invention.

1. A system for estimating a level of interoception in a subject, the system comprising:
a heartbeat sensor for sensing the subject's heartbeat as manifested locally to the sensor;
a user interface for receiving input from the subject; and
a processor configured to:
(i) receive information representing a timing of sensed heartbeats,
(ii) form, in dependence on the received information, a periodic output having a same frequency as the sensed heartbeats,
(iii) present the periodic output to the subject,
(iv) receive input of a first type from the user interface, and
(v) in dependence on the input of the first type, adjust a phase with which the periodic output is presented to the subject.

2. The system of claim 1, wherein the processor is further configured to:
(vi) receive input of a second type from the user interface, and
(vii) in response to the input of the second type, store a current timing offset between the sensed heartbeats and periodic elements of the periodic output.

3. The system of claim 1, wherein the processor is further configured to:
determine, prior to presenting the periodic output to the subject, a random offset from the sensed heartbeats; and
cause the periodic output to be initially presented to the subject at a phase corresponding to an offset between the sensed heartbeats and periodic elements of the periodic output that is equal to the random offset.

4. The system of claim 1, wherein the periodic output comprises a sound comprising periodic elements.

5. The system of claim 4, wherein the periodic elements comprise tones.

6. The system of claim 1, wherein the user interface provides a first user interface element configured to be adjusted continuously by the subject and the first user interface element forms the input of the first type.

7. The system of claim 6, wherein the first user interface element comprises a wheel.

8. The system of claim 6, wherein the user interface provides a second user interface element comprising a button.

9. The system of claim 2, wherein the processor is further configured to repeatedly perform steps (i)-(vii) and to form an estimate of a consistency of the stored timing offsets.

10. The system of claim 1, the system being configured to form an estimate of the subject's level of interoception in dependence on one or both of (i) absolute values of one or more of the stored timing offsets and (ii) an estimated consistence of the stored timing offsets.

11. The system of claim 1, the system being configured to assess an identity of the subject in dependence on one or both of (i) absolute values of one or more of the stored timing offsets and (ii) an estimated consistence of the stored timing offsets.

12. A method for estimating a level of interoception in a subject, the method comprising the steps of:
sensing the timing of the subject's heartbeats as manifested locally to a sensor;
forming, in dependence on the received information, a periodic output having a same frequency as the sensed heartbeats;
presenting the periodic output to the subject;
receiving input of a first type from a user interface; and
in dependence on the input of a first type, adjusting a phase with which the periodic output is to presented to the subject.
